(19) United States
(12) Patent Application Publication
(10) Pub. No.: US 2010/0215783 A1
(43) Pub. Date: Aug. 26, 2010

(54) Antioxidant Compositions, Beverage Formulations Thereof, and Methods of Increasing Antioxidant Capability

(75) Inventor: Peter S. McNeary, Brattleboro, VT (US)

Correspondence Address: DOWNS RACHLIN MARTIN PLLC, 199 MAIN STREET, P O BOX 190, BURLINGTON, VT, 05402-0190, US

(73) Assignee: NUTRAGENESIS, LLC, Brattleboro, VT (US)

(21) Appl. No.: 12/710,843

(22) Filed: Feb. 23, 2010

Related U.S. Application Data
(60) Provisional application No. 61/154,647, filed on Feb. 23, 2009.

(51) Int. Cl. A61K 36/45; A61K 36/736; A61K 36/752; A61K 36/87; A61K 31/34

(52) U.S. Cl. 424/732; 424/777; 424/735; 424/736; 424/766; 514/474

(57) ABSTRACT

Compositions including synergistic combinations of ascorbic acid, Indian gooseberry, and additional components for increasing the antioxidant capability of certain target components, such as superfruits, fruits, and other low and/or high antioxidant containing substances. Particular combinations of ascorbic acid, Indian gooseberry extract, one or more citrus bioflavanoids, and one or more fatty acids added to various target components synergistically increases the antioxidant capability of the overall composition over expected values.

### RELATED APPLICATION DATA

This application claims the benefit of priority of U.S. Provisional Patent Application Ser. No. 61/154,647, filed Feb. 23, 2009, and titled “Antioxidant Compositions, Beverage Formulations Thereof, And Methods Of Increasing Antioxidant Capability,” which is incorporated by reference herein in its entirety.

### FIELD OF THE INVENTION

The present invention generally relates to the field of antioxidant activity. In particular, the present invention is directed to antioxidant compositions, beverage formulations thereof, and methods of increasing antioxidant capability.

### BACKGROUND

Antioxidant compounds are frequently used in dietary supplements to fight against oxidative impact of environmental, natural, and other influences. Some common antioxidants include Vitamin E and ascorbic acid, also known as Vitamin C. Vitamin E and Vitamin C also appear naturally in fruits and vegetables. Although Vitamin C has antioxidative properties, U.S. Pat. No. 6,362,167 sets forth the pro-oxidant properties of Vitamin C in certain environments.

Emblica officinalis (Indian gooseberry) is also known to have antioxidative properties. Some of the antioxidative properties of Emblica officinalis are described in U.S. Pat. No. 6,124,268.

Superfruits are utilized and marketed in food and beverage products, such as juices, for nutrient and antioxidant qualities. Exemplary superfruits include acai, blueberry, pomegranate, cranberry, goji, and others.

### SUMMARY OF THE DISCLOSURE

In one embodiment, a composition having increased antioxidant capability is provided. The composition includes a synergistically effective combination of an Indian gooseberry extract; ascorbic acid; and a target component, wherein the Indian gooseberry extract, the ascorbic acid, and the target component are present in amounts effective to synergistically increase the antioxidant capability of the target component.

In another embodiment, an antioxidant composition is provided. The composition includes about 19 wt. % to about 76 wt. % ascorbic acid; about 0.3 wt. % to about 1.2 wt. % one or more citrus bioflavanoids; about 0.1 wt. % to about 0.4 wt. % one or more fatty acids; and about 12 wt. % to about 48 wt. % one or more low molecular weight hydrolysable tannins of an extract of Indian gooseberry.

In yet another embodiment, a method of synergistically increasing the antioxidant capability of a target component is provided. The method includes providing a target component; and adding a synergistically effective amount of an extract of Indian gooseberry, ascorbic acid, and one or more additional components to the target component such that the antioxidant capability of the target component is synergistically increased.

### DETAILED DESCRIPTION

In one embodiment, an antioxidant composition is provided. The antioxidant composition includes a combination of an extract of Emblica officinalis (Indian gooseberry) and ascorbic acid (Vitamin C). In one exemplary aspect, an antioxidant composition according to the present disclosure is effective to synergistically increase the antioxidant effect of any one or more of a variety of superfruit extracts to an unexpected degree. In another exemplary aspect, an antioxidant composition according to the present disclosure is effective to synergistically increase the antioxidant effect of any one or more of a variety of beverages.

An antioxidant composition according to the present disclosure may include a synergistically effective amount of an extract of Indian gooseberry. In one example, an antioxidant composition according to the present disclosure includes about 20 wt. % extract of Indian gooseberry to about 80 wt % extract of Indian gooseberry. In another example, an antioxidant composition according to the present disclosure includes about 30 wt % extract of Indian gooseberry to about 40 wt % extract of Indian gooseberry. In yet another example, an antioxidant composition according to the present disclosure includes about 37.5 wt. % extract of Indian gooseberry. In still another example, an antioxidant composition includes at least about 37.5 wt. % extract of Indian gooseberry. In still yet another example, an antioxidant composition according to the present disclosure includes about 20 wt. % extract of Indian gooseberry. In a further example, an antioxidant composition includes at least about 20 wt. % extract of Indian gooseberry. In still a further example, an antioxidant composition according to the present disclosure includes about 80 wt. % extract of Indian gooseberry. In yet a further example, an antioxidant composition includes at least about 80 wt. % extract of Indian gooseberry.

An extract of Indian gooseberry may be derived in a variety of ways. One example of a method of deriving an extract of Indian gooseberry is disclosed in U.S. Pat. No. 6,124,268, the disclosure of which is incorporated herein in its entirety. An extract of Indian gooseberry may include one or more low molecular weight hydrolysable tannins. Examples of such low molecular weight hydrolysable tannins include, but are not limited to, Emblicanin A, Emblicanin B, Emblicanin oligomers and any combinations thereof. Emblicanin A and Emblicanin B are examples of gallic/ellagic acid derivatives of 2-keto-glucono-δ-lactone. In one example, an extract of Indian gooseberry included in an antioxidant composition of the present disclosure includes Emblicanin A and Emblicanin B.

In one example, an antioxidant composition according to the present disclosure includes about 12 wt. % to about 48 wt. % low molecular weight hydrolysable tannins of an extract of Indian gooseberry. In another example, an antioxidant composition according to the present disclosure includes about 20 wt. % to about 25 wt. % low molecular weight hydrolysable tannins of an extract of Indian gooseberry. In still another example, an antioxidant composition according to the present disclosure includes about 22.5 wt. % low molecular weight hydrolysable tannins of an extract of Indian gooseberry. In yet another example, an antioxidant composition according to the present disclosure includes at least about 22.5 wt. % low molecular weight hydrolysable tannins of an extract of Indian gooseberry. In still yet another example, an antioxidant composition according to the present disclosure includes about 12 wt. % low molecular weight hydrolysable tannins of an extract of Indian gooseberry. In a further example, an antioxidant composition according to the present disclosure includes at least about 12 wt. % low molecular weight hydrolysable tannins of an extract of Indian gooseberry. In still a further example, an antioxidant composition according to the present disclosure includes about 48 wt. % low molecular weight hydrolysable tannins of an extract of Indian gooseberry. In yet a further example, an antioxidant composition according to the present disclosure includes at least about 48 wt. % low molecular weight hydrolysable tannins of an extract of Indian gooseberry.

One example of an extract of Indian gooseberry includes about 35 wt. % to about 55 wt. % Emblicanin A and Emblicanin B, about 4 wt. % to about 15 wt. % Punigluconin (2,3-di-O-galloyl-4,6-(S)-hexahydroxy-diphenoylgluconic acid), about 10 wt. % to about 20 wt. % Pedunculagin (2,3,4,6-bis-(S)-hexahydroxydiphenoyl-D-glucose), Rutin (flavanol-3-glycoside), and about 10 wt. % to about 30 wt. % low to medium molecular weight gallo-ellagi tannoids. In another example, an extract of Indian gooseberry includes about 27 wt. % Emblicanin-A, about 23 wt. % Emblicanin-B, about 8 wt. % Punigluconin, about 14 wt. % Pedunculagin, about 10 wt. % Rutin, and about 10 wt. % to about 30 wt. % gallo-ellagitannoids. In yet another example, an extract of Indian gooseberry includes a commercially available extract of Indian gooseberry available from Natreon of New Brunswick, N.J. under the tradename CAPROS.

The molecular structure of Emblicanin-A is:

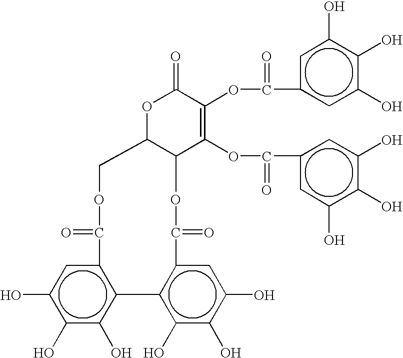

The molecular structure of Emblicanin-B is:

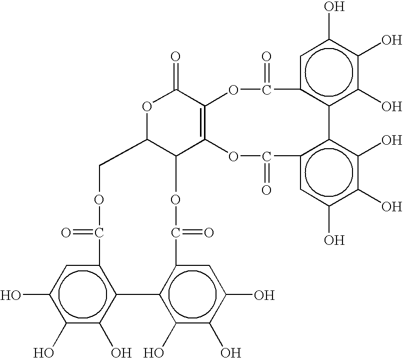

The molecular structure of Punigluconin is:

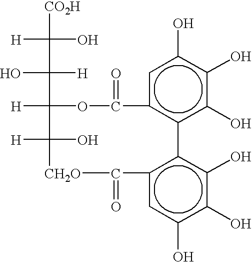

The molecular structure of Pedunculagin is:

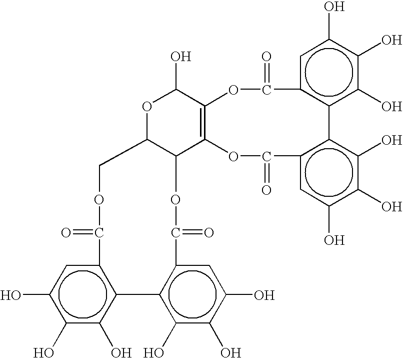

The molecular structure of Rutin is:

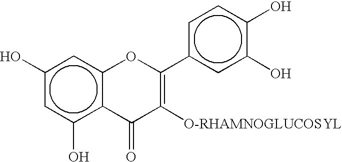

Ascorbic acid present in an antioxidant composition of the present disclosure may be part of an ascorbic acid preparation having additional additive components. Examples of such additive components include, but are not limited to, one or more citrus bioflavanoids, one or more fatty acids, and any combinations thereof. In one example, an ascorbic acid preparation is commercially available from Innovation Laboratories, Inc. of Mount Sinai, N.Y. under the tradename PUREWAY-C.

One example of an ascorbic acid preparation includes at least about 95 wt. % ascorbic acid, at least about 1.5 wt. % citrus bioflavanoids, and at least about 0.5 wt. % fatty acids. An antioxidant composition according to the present disclosure may include a synergistically effective amount of an ascorbic acid preparation. In one example, an antioxidant composition according to the present disclosure includes about 20 wt. % to about 80 wt % of an ascorbic acid preparation. In another example, an antioxidant composition according to the present disclosure includes about 30 wt % to about 40 wt % of an ascorbic acid preparation. In yet another example, an antioxidant composition according to the present disclosure includes about 37.5 wt. % of ascorbic acid preparation. In still another example, an antioxidant composition includes at least about 37.5 wt. % of an ascorbic acid preparation. In still yet another example, an antioxidant composition according to the present disclosure includes about 20 wt. % of an ascorbic acid preparation. In a further example, an antioxidant composition includes at least about 20 wt. % of an ascorbic acid preparation. In still a further example, an antioxidant composition according to the present disclosure includes about 80 wt. % of an ascorbic acid preparation. In yet a further example, an antioxidant composition includes at least about 80 wt. % an ascorbic acid preparation.

As discussed above, the antioxidant composition of the present disclosure includes ascorbic acid. In one example, an antioxidant composition according to the present disclosure includes about 19 wt. % to about 76 wt. % ascorbic acid. In another example, an antioxidant composition according to the present disclosure includes about 55 wt. % to about 65 wt. % ascorbic acid. In still another example, an antioxidant composition according to the present disclosure includes about 59.4 wt. % ascorbic acid. In yet another example, an antioxidant composition according to the present disclosure includes at least about 59.4 wt. % ascorbic acid. In still yet another example, an antioxidant composition according to the present disclosure includes about 19 wt. % ascorbic acid. In a further example, an antioxidant composition according to the present disclosure includes at least about 19 wt. % ascorbic acid. In still a further example, an antioxidant composition according to the present disclosure includes about 76 wt. % ascorbic acid. In yet a further example, an antioxidant composition according to the present disclosure includes at least about 76 wt. % ascorbic acid.

An antioxidant composition of the present disclosure may also include one or more citrus bioflavanoids. In one example, an antioxidant composition according to the present disclosure includes about 0.3 wt. % to about 1.2 wt. % citrus bioflavanoids. In another example, an antioxidant composition according to the present disclosure includes about 0.8 wt. % to about 1 wt. % citrus bioflavanoids. In still another example, an antioxidant composition according to the present disclosure includes about 0.9 wt. % citrus bioflavanoids. In yet another example, an antioxidant composition according to the present disclosure includes at least about 0.9 wt. % citrus bioflavanoids. In still yet another example, an antioxidant composition according to the present disclosure includes about 0.3 wt. % citrus bioflavanoids. In a further example, an antioxidant composition according to the present disclosure includes at least about 0.3 wt. % citrus bioflavanoids. In still a further example, an antioxidant composition according to the present disclosure includes about 1.2 wt. % citrus bioflavanoids. In yet a further example, an antioxidant composition according to the present disclosure includes at least about 1.2 wt. % citrus bioflavanoids.

An antioxidant composition of the present disclosure may also include one or more fatty acids. In one example, an antioxidant composition according to the present disclosure includes about 0.1 wt. % to about 0.4 wt. % fatty acids. In another example, an antioxidant composition according to the present disclosure includes about 0.2 wt. % to about 0.4 wt. % fatty acids. In still another example, an antioxidant composition according to the present disclosure includes about 0.3 wt. % fatty acids. In yet another example, an antioxidant composition according to the present disclosure includes at least about 0.3 wt. % fatty acids. In still yet another example, an antioxidant composition according to the present disclosure includes about 0.1 wt. % fatty acids. In a further example, an antioxidant composition according to the present disclosure includes at least about 0.1 wt. % fatty acids. In still a further example, an antioxidant composition according to the present disclosure includes about 0.4 wt. % fatty acids. In yet a further example, an antioxidant composition according to the present disclosure includes at least about 0.4 wt. % fatty acids.

### Examples 1 to 3

An exemplary antioxidant composition was prepared. In this example, an ascorbic acid preparation was combined with an extract of Indian gooseberry at different ratios. The example ascorbic acid preparation included at least about 95 wt. % ascorbic acid, at least about 1.5 wt. % citrus bioflavanoids, at least about 0.5 wt. % fatty acids, and at least about 1 wt. % water. The example Indian gooseberry extract included at least about 60 wt. % low molecular weight hydrolysable tannins, at least about 6 wt. % sulfated ash, and at least about 6 wt. % water. As set forth in Table 1 below, the ascorbic acid preparation and extract of Indian gooseberry were combined in Experimental Example 1 in ratios of 5:3. It is contemplated that an ascorbic acid preparation, an extract of Indian gooseberry, and optionally one or more of a citrus bioflavanoid, a fatty acid, and a sulfated ash, may be combined in other ratios of combination. As exhibited in Example 1, it is possible, but not a requirement, that one or more citrus bioflavanoids, one or more fatty acids, and/or one or more sulfated ashes may be included as part of one or more of an ascorbic acid preparation and an extract of Indian gooseberry. In another example, an ascorbic acid preparation and an extract of Indian gooseberry may be combined in a ratio of 4:1. Table 1 illustrates an example (Example 2) in which an ascorbic acid preparation and an extract of Indian gooseberry may be combined in a 4:1 ratio. In yet another example, an ascorbic acid preparation and an extract of Indian gooseberry may be combined in a ratio of 1:4. Table 1 illustrates an example (Example 3) in which an ascorbic acid preparation and an extract of Indian gooseberry may be combined in a 1:4 ratio.

TABLE 1
Data for Three Exemplary Combinations of Ascorbic
Acid Preparation and Extract of Indian Gooseberry

| | | | Ratio of Ascorbic | |
|---|---|---|---|---|
| | Ascorbic | Extract of | Acid Prep | |
| | Acid | Indian | to Extract | Antioxidant |
| | Preparation | Gooseberry | of Indian | Composition |
| Analytical test | (wt. %) | (wt %) | Gooseberry | (wt. %) |
| (Example 1) | | | | |
| Vitamin C | 95 | 0 | 5:3 | 59.4 |
| Citrus Bioflavonoids | 1.5 | 0 | 5:3 | 0.9 |
| Fatty Acids | 0.5 | 0 | 5:3 | 0.3 |
| LMW hydrolyzable tannins | 0 | 60 | 5:3 | 22.5 |
| Water (Karl Fischer) | 1 | 6 | 5:3 | 2.9 |
| Sulfated Ash | 0 | 6 | 5:3 | 2.3 |
| (Example 2) | | | | |
| Vitamin C | 95 | 0 | 4:1 | 76 |
| Citrus Bioflavonoids | 1.5 | 0 | 4:1 | 1.2 |
| Fatty Acids | 0.5 | 0 | 4:1 | 0.4 |
| LMW hydrolyzable tannins | 0 | 60 | 4:1 | 12 |
| Water (Karl Fischer) | 1 | 6 | 4:1 | 2 |
| Sulfated Ash | 0 | 6 | 4:1 | 1.2 |
| (Example 3) | | | | |
| Vitamin C | 95 | 0 | 1:4 | 19 |
| Citrus Bioflavonoids | 1.5 | 0 | 1:4 | 0.3 |
| Fatty Acids | 0.5 | 0 | 1:4 | 0.1 |
| LMW hydrolyzable tannins | 0 | 60 | 1:4 | 48 |
| Water (Karl Fischer) | 1 | 6 | 1:4 | 5 |
| Sulfated Ash | 0 | 6 | 1:4 | 4.8 |

It has been unexpectedly found that combination of an antioxidant composition of the present disclosure, as discussed above, with a superfruit (e.g., a superfruit extracts) and/or a fruit produces a synergistic increase in the level of antioxidant activity of the combination. In one exemplary aspect, an antioxidant composition of the present disclosure may be utilized to increase the antioxidant effect of a beverage (e.g., hot, cold, etc.) including a superfruit and/or fruit (e.g. as an extract thereof) [or one or more other target component as discussed below]. Other uses of an antioxidant composition of the present disclosure include, but are not limited to, increase the antioxidant effect of a nutritional product (e.g., a soup, a vitamin enriched granola bar, a nutritional bar, whole grain bread), a dietary supplement, a food product (e.g., hot, cold, etc.), a confectionary, an oil, a meal replacement, a cereal, a baked good, a candy, a gum, a lozenge, and any combinations thereof. In one example, a synergistically increased antioxidant composition is in the form of a beverage.

In one embodiment, a synergistically increased antioxidant composition includes a combination of an antioxidant composition as discussed above (including an extract of Indian gooseberry, ascorbic acid, and optionally other components), and one or more superfruits and/or fruits. Example superfruits and fruits include, but are not limited to, pomegranate, blueberry, cranberry, goji, acai, mangosteen, gac, noni, guarana, seabuckthorn, apple, orange, tomato, cranberry, concord grape, white grape, muscadine grape, grapefruit, strawberry, raspberry, blackberry, lemon, lime, key lime, kiwi, tangerine, banana, sweet cherry, black cherry, tart cherry, peach, plum, nectarine, pear, black currant, elderberry, apricot, mango, pineapple, passionfruit, yumberry, watermelon, cantaloupe, muskmelon, honeydew, starfruit, dragonfruit, damson plum, prune, raisin, lychee, schisandra, chokeberry, lingonberry, acerola, baobab, bilberry, camu camu, cupuacu, date, fig, durian, red guava, longan, papaya, Saskatoon berry, tamarind, yuzu, olive, and any combinations thereof. A superfruit and/or a fruit may be present in a variety of forms including, but not limited to, an extract, a juice, a powder, a concentrate, a sauce, a preserve, and any combinations thereof. A synergistically increased antioxidant composition may also include one or more citrus bioflavanoids, one or more fatty acids, sulfated ash, and any combinations thereof.

In one example, a synergistically increased antioxidant composition includes a combination of an antioxidant composition as described above with an extract of pomegranate. In another example, a synergistically increased antioxidant composition includes a combination of an antioxidant composition as described above with an extract of blueberry. In still another example, a synergistically increased antioxidant composition includes a combination of an antioxidant composition as described above with an extract of cranberry. In yet another example, a synergistically increased antioxidant composition includes a combination of an antioxidant composition as described above with an extract of goji. In still yet another example, a synergistically increased antioxidant composition includes a combination of an antioxidant composition as described above with an extract of acai.

In another embodiment, a synergistically increased antioxidant composition includes a combination of an antioxidant composition as discussed above (including an extract of Indian gooseberry, ascorbic acid, and optionally other components), and one or more juices. Examples of a juice include, but are not limited to, filtered apple juice, unfiltered apple juice, cranberry juice, orange juice, tomato juice, pomegranate juice, blueberry juice, goji juice, acai juice, white grape, Concord grape, a juice of a fruit and/or superfruit as discussed above, and any combinations thereof. A synergistically increased antioxidant composition may also include one or more citrus bioflavanoids, one or more fatty acids, sulfated ash, and any combinations thereof.

In one example, a synergistically increased antioxidant composition includes a combination of an antioxidant composition as described above with filtered apple juice. In another example, a synergistically increased antioxidant composition includes a combination of an antioxidant composition as described above with unfiltered apple juice. In yet another example, a synergistically increased antioxidant composition includes a combination of an antioxidant composition as described above with cranberry juice. In still another example, a synergistically increased antioxidant composition includes a combination of an antioxidant composition as described above with orange juice. In still yet another example, a synergistically increased antioxidant composition includes a combination of an antioxidant composition as described above with tomato juice. In a further example, a synergistically increased antioxidant composition includes a combination of an antioxidant composition as described above with pomegranate juice. In still a further example, a synergistically increased antioxidant composition includes a combination of an antioxidant composition as described above with blueberry juice. In yet a further example, a synergistically increased antioxidant composition includes a combination of an antioxidant composition as described above with goji juice. In still yet a further example, a synergistically increased antioxidant composition includes a combination of an antioxidant composition as described above with acai juice.

In yet another embodiment, a synergistically increased antioxidant composition includes a combination of an antioxidant composition as discussed above (including an extract of Indian gooseberry, ascorbic acid, and optionally other components), and one or more teas. Example teas include, but are not limited to, green tea, black tea, white tea, red tea (e.g., Roobois), and any combinations thereof. The synergistically increased antioxidant composition may also include one or more citrus bioflavonoids, one or more fatty acids, sulfated ash, and any combinations thereof. In one example, a synergistically increased antioxidant composition includes a combination of an antioxidant composition as described above with a green tea. In another example, a synergistically increased antioxidant composition includes a combination of an antioxidant composition as described above with a black tea. In yet another example, a synergistically increased antioxidant composition includes a combination of an antioxidant composition as described above with a white tea. In still another example, a synergistically increased antioxidant composition includes a combination of an antioxidant composition as described above with a red tea.

In still yet another embodiment, a synergistically increased antioxidant composition includes a combination of an antioxidant composition as discussed above and another target component. Examples of other target components include, but are not limited to, a vegetable, a medicinal botanical, a chocolate, a cocoa, a coffee, a chicory, a mushroom, a grain, a legume, a nut, a seed, an oil, a culinary spice, a grass, an algae, and any combinations thereof. Example vegetables include, but are not limited to, a carrot, an artichoke, an arugula, an asparagus, a broccoli, a cabbage, a cauliflower, a celery, a cucumber, a beet, a swiss chard, a collard green, a mustard green, a kale, a brussel sprout, a spinach, a turnip, a lettuce, an okra, a pepper, a bean, a pea, a squash, a watercress, a tomato, a corn, a pumpkin, an eggplant, a peanut, an onion, a garlic, a leek, a parsnip, a turnip, a rutabaga, a radish, a potato, a sweet potato, a yam, a horseradish, and any combinations thereof. Exemplary forms of a vegetable include, but are not limited to, an extract, a powder, a juice, a concentrate, a sauce, a preserve, and any combinations thereof. Example medicinal botanicals include, but are not limited to, ashwagandha, holy basil, ginseng, rhodiola, chamomile, lemon balm, gotu kola, bacopa, schisandra, and any combinations thereof. Exemplary forms of a medicinal botanical include, but are not limited to, a dried botanical, an extract, a powder, a concentrate, and any combinations thereof. Example mushrooms include, but are not limited to, a portabella, a white button top, a maitake, an oyster, a shiitake, a reishi, a cordyceps, a brazilian mushroom, and any combinations thereof. Exemplary forms of a mushroom include, but are not limited to, a dried mushroom, an extract, a powder, a concentrate, and any combinations thereof. Example grains include, but are not limited to, corn, purple corn, blue corn, black corn, wheat, rice, rye, oat, barley, millet, quinoa, buckwheat, sorghum, wild rice, tapioca, and any combinations thereof. Example legumes include, but are not limited to, soybean, lentil, kidney, pinto, black, chickpea, faba, lima, peas, black-eyed pea, split pea, butter, navy, peanut, and any combinations thereof. Example nuts include, but are not limited to, almond, cashew, peanut, macadamia, pecan, filbert, brazil, pistachio, walnut, pine, coconut, chestnut, kola, and any combinations thereof. Example seeds include, but are not limited to, grape, sunflower, quinoa, salba, sesame, pumpkin, flax, borage, evening primrose, grain amaranth, and any combinations thereof. Example oils include, but are not limited to, olive, canola, sunflower, coconut, safflower, soybean, grapeseed, palm, sesame, cottonseed, peanut, corn, walnut, almond, and any combinations thereof. Example culinary spices include, but are not limited to, ginger, turmeric, rosemary, oregano, basil, and any combinations thereof. Exemplary forms of a culinary spice include, but are not limited to, a dried spice, an extract, a powder, a concentrate, and any combinations thereof. Examples of a grass and/or an algae include, but are not limited to, alfalfa, spirulina, chlorella, and any combinations thereof. Exemplary forms of a grass and/or algae include, but are not limited to, dried, an extract, a powder, a concentrate, and any combinations thereof.

Although embodiments and examples discussed herein may describe a combination of an antioxidant composition (including an extract of Indian gooseberry, ascorbic acid, and optionally other components) and one or more other components (e.g., a superfruit, a fruit, a vegetable, a tea, etc.), it is contemplated that the components of the resultant combination may be combined in any fashion (e.g., not by first creating an antioxidant composition that is then combined with other components). In one example, the components discussed above may be combined together into a resultant synergistically increased composition.

In one implementation, a synergistically increased antioxidant composition as discussed above includes a resultant synergism over the expected antioxidant effect for a composition including a combination of an antioxidant composition according to the present disclosure (including an extract of Indian gooseberry, ascorbic acid, and optionally one or more citrus bioflavanoids, one or more fatty acids, and/or one or more sulfated ashes) and target component as discussed in the examples and embodiments herein (e.g., a superfruit, a fruit, a tea, a vegetable, a medicinal botanical, a coffee, a chocolate, a cocoa, a chicory, a mushroom, a grain, a legume, a nut, a seed, an oil, a culinary spice, a grass, and/or an algae). In one such implementation, synergism is measured by an ABTS radical decolorisation assay as set forth in Experimental Example 4 below. In one example, a synergistically increased antioxidant composition has an ABTS degree of synergism of at least about 1.2 for a 1:1 (w/w) combination of antioxidant composition and target component. In another example, a synergistically increased antioxidant composition has an ABTS degree of synergism of at least about 1.4 for a 1:1 (w/w) combination of antioxidant composition and target component. In still another example, a synergistically increased antioxidant composition has an ABTS degree of synergism of at least about 1.7 for a 1:1 (w/w) combination of antioxidant composition and target component. In yet another example, a synergistically increased antioxidant composition has an ABTS degree of synergism of at least about 2 for a 1:1 (w/w) combination of antioxidant composition and target component. In still yet another example, a synergistically increased antioxidant composition has an ABTS degree of synergism of at least about 3 for a 1:1 (w/w) combination of antioxidant composition and target component. In a further example, a synergistically increased antioxidant composition has an ABTS degree of synergism of at least about 4 for a 1:1 (w/w) combination of antioxidant composition and target component. In still a further example, a synergistically increased antioxidant composition has an ABTS degree of synergism of at least about 5 for a 1:1 (w/w) combination of antioxidant composition and target component.

In other examples, an ABTS assay may also be utilized to measure the degree of synergism over the expected antioxidant effect for a composition including a combination of an antioxidant composition and a target component that is in the form of a juice. In one example, a synergistically increased antioxidant composition has an ABTS degree of synergism of at least about 0.6 for a combination of 200 mg of an antioxidant composition as discussed above to 8 ounces of a beverage. In another example, a synergistically increased antioxidant composition has an ABTS degree of synergism of at least about 0.7 for a combination of 200 mg of an antioxidant composition as discussed above to 8 ounces of a beverage. In yet another example, a synergistically increased antioxidant composition has an ABTS degree of synergism of at least about 1 for a combination of 200 mg of an antioxidant composition as discussed above to 8 ounces of a beverage. In still another example, a synergistically increased antioxidant composition has an ABTS degree of synergism of at least about 1.1 for a combination of 200 mg of an antioxidant composition as discussed above to 8 ounces of a beverage. In still yet another example, a synergistically increased antioxidant composition has an ABTS degree of synergism of at least about 1.2 for a combination of 200 mg of an antioxidant composition as discussed above to 8 ounces of a beverage.

In another such implementation, synergism is measured by a DPPH radical decolorisation assay as set forth in Experimental Example 4 below. In one example, a synergistically increased antioxidant composition has a DPPH degree of synergism of at least about 1.1 for a 1:1 (w/w) combination of antioxidant composition and target component. In another example, a synergistically increased antioxidant composition has a DPPH degree of synergism of at least about 1.4 for a 1:1 (w/w) combination of antioxidant composition and target component. In still another example, a synergistically increased antioxidant composition has a DPPH degree of synergism of at least about 1.5 for a 1:1 (w/w) combination of antioxidant composition and target component. In yet another example, a synergistically increased antioxidant composition has an a DPPH degree of synergism of at least about 2 for a 1:1 (w/w) combination of antioxidant composition and target component. In still yet another example, a synergistically increased antioxidant composition has a DPPH degree of synergism of at least about 3 for a 1:1 (w/w) combination of antioxidant composition and target component. In a further example, a synergistically increased antioxidant composition has a DPPH degree of synergism of at least about 16 for a 1:1 (w/w) combination of antioxidant composition and target component. In still a further example, a synergistically increased antioxidant composition has a DPPH degree of synergism of at least about 20 for a 1:1 (w/w) combination of antioxidant composition and target component. In yet a further example, a synergistically increased antioxidant composition has a DPPH degree of synergism of at least about 51 for a 1:1 (w/w) combination of antioxidant composition and target component. In still yet a further example, a synergistically increased antioxidant composition has a DPPH degree of synergism of at least about 25 for a 1:1 (w/w) combination of antioxidant composition and target component.

### Experimental Example 4

An extract of pomegranate (having 40% bioactives) was combined with dextrin white in ratios of 1:1 weight to weight (w/w) and 1:3 (w/w) to achieve samples with bioactive concentrations of 40%, 20% and 10%, respectively.

An ascorbic acid preparation was prepared according to Example 1 above. An extract of Indian gooseberry was prepared according to Example 1 above. An antioxidant composition was prepared by a combination of 125 mg of the ascorbic acid preparation to 75 mg of the extract of Indian gooseberry (5:3 w/w).

The antioxidant effect of each of the extract of Indian gooseberry, ascorbic acid preparation, and antioxidant composition with each of the extracts of pomegranate was tested. Each of the extract of Indian gooseberry, ascorbic acid preparation, and the antioxidant composition were combined separately with samples of the three pomegranate extracts by dissolving in distilled water separately and mixing to obtain a final concentration of 1 mg/ml of the mixture containing the two components in 1:1 w/w ratio. Individual components and the combined mixtures were assayed for their antioxidant profiles in vitro.

Synergism was calculated from the following formula—

$$Degree of synergism = \frac{Expected {IC}_{50} of the mixture}{Observed {IC}_{50} of the mixture}$$

$$Expected {IC}_{50} of the mixture = Mean of the {IC}_{50} values of the individual components$$

Since, in case of synergism, the observed antioxidant profile in terms of IC₅₀ of the mixture would be lower than the expected IC₅₀ value, the degree of synergism would be greater than 1. Higher values denote profound synergism. The terms “synergy” and “synergism” are used interchangeably herein.

ABTS and DPPH radical decolorisation assays of the individual samples were performed and the antioxidant profile has been provided for each in Table 2 and 3. Synergism data is provided for the extract of Indian gooseberry, ascorbic acid preparation, and antioxidant composition each with each of the pomegranate extracts below in Tables 4 to 9. A profound synergism was observed between the antioxidant composition and each of the three pomegranate extracts with 10%, 20%, and 40% concentrations of bioactives (mixed in 1:1 w/w ratio). See Tables 8 and 9. The ABTS radical cation decolorisation assay was performed according to the method of Pellegrini et al. (Pellegrini, N., Serafini, M., Colombi, B., Del Rio, D., Salvatore, S., Bianchi, M. And Brighenti, F. 2003. J. Nutr., 133, 2812-2819), which is incorporated herein by reference in its entirety. Briefly, neutralization of the radical and reduction of color at 734 nm by the antioxidants was measured and calculated with respect to the control. The results were expressed as IC₅₀ of the samples. The DPPH radical decolorisation assay was performed according to the method of Gulcin et al. (Gulcin, I., Kufrevioglu, O, I., Oktay, M. and Buyukokuroglu, M. E. 2004. J. Ethnopharmacol., 90(2-3), 205-215), which is incorporated herein by reference in its entirety. Briefly, neutralization of the radical and reduction of color at 517 nm by the antioxidants was measured and calculated with respect to the control. The results were expressed as IC₅₀ of the samples.

TABLE 2
IC₅₀ values (in micrograms(μg)/milliliter(ml))
of different extracts in ABTS radical cation decolorisation assay

| Sample | IC₅₀ values |
|---|---|
| Antioxidant composition | 2.47 ± 0.36 |
| Extract of Indian gooseberry | 3.03 ± 0.19 |
| Ascorbic acid preparation | 2.10 ± 0.25 |
| 40% Pomegranate | 4.92 ± 0.26 |
| 20% Pomegranate | 10.31 ± 0.88 |
| 10% Pomegranate | 18.38 ± 0.66 |

TABLE 3
IC₅₀ values (in micrograms(μg)/millileter(ml))
of different extracts in DPPH radical decolorisation assay

| Sample | IC₅₀ values |
|---|---|
| Antioxidant composition | 8.67 ± 0.22 |
| Extract of Indian gooseberry | 10.77 ± 0.86 |
| Ascorbic acid preparation | 6.91 ± 0.57 |
| 40% Pomegranate | 12.19 ± 0.79 |
| 20% Pomegranate | 19.43 ± 0.88 |
| 10% Pomegranate | 35.72 ± 2.20 |

TABLE 4
Degree of synergism of mixtures of Extract of Indian gooseberry and three pomegranate
extracts in ABTS radical cation decolorisation assay

| | IC₅₀ values of | | | | |
|---|---|---|---|---|---|
| | individual | | | | |
| | components | | | | |
| | Extract of | | | | |
| | Indian | Pomegranate | Expected | | Degree of |
| | gooseberry | Sample | IC₅₀ values | Observed | synergism |
| Sample | [A] | [B] | [A + B]/2 | IC₅₀ values | [exp./obs] |
| Extract of Indian | 3.03 ± 0.19 | 4.92 ± 0.26 | 3.98 | 3.19 ± 0.64 | 1.25 |
| gooseberry + 40% | | | | | |
| Pomegranate in | | | | | |
| 1:1 w/w ratio | | | | | |
| Extract of Indian | 3.03 ± 0.19 | 10.31 ± 0.88 | 6.67 | 4.54 ± 0.84 | 1.47 |
| gooseberry + 20% | | | | | |
| Pomegranate in | | | | | |
| 1:1 w/w ratio | | | | | |
| Extract of Indian | 3.03 ± 0.19 | 18.38 ± 0.66 | 10.71 | 6.22 ± 0.91 | 1.72 |
| gooseberry + 10% | | | | | |
| Pomegranate in | | | | | |
| 1:1 w/w ratio | | | | | |

TABLE 5
Degree of synergism of mixtures of Extract of Indian gooseberry and
three pomegranate extracts in DPPH radical cation decolorisation assay

| | IC₅₀ values of individual | | | | |
|---|---|---|---|---|---|
| | components | | | | |
| | Extract of | | | | |
| | Indian | Pomegranate | Expected | | Degree of |
| | gooseberry | Sample | IC₅₀ values | Observed | synergism |
| Sample | [A] | [B] | [A + B]/2 | IC₅₀ values | [exp./obs] |
| Extract of Indian | 10.77 ± 0.86 | 12.19 ± 0.79 | 11.48 | 10.41 ± 0.78 | 1.10 |
| gooseberry + 40% | | | | | |
| Pomegranate in | | | | | |
| 1:1 w/w ratio | | | | | |
| Extract of Indian | 10.77 ± 0.86 | 19.43 ± 0.88 | 15.10 | 12.48 ± 0.71 | 1.21 |
| gooseberry + 20% | | | | | |
| Pomegranate in | | | | | |
| 1:1 w/w ratio | | | | | |
| Extract of Indian | 10.77 ± 0.86 | 35.72 ± 2.20 | 23.25 | 17.12 ± 0.85 | 1.36 |
| gooseberry + 10% | | | | | |
| Pomegranate in | | | | | |
| 1:1 w/w ratio | | | | | |

TABLE 6
Degree of synergism of mixtures of Ascorbic acid preparation and three pomegranate
extracts in ABTS radical cation decolorisation assay

| | IC₅₀ values | | | | |
|---|---|---|---|---|---|
| | of individual | | | | |
| | components | | | | |
| | Ascorbic | | | | |
| | acid | Pomegranate | Expected | | Degree of |
| | preparation | Sample | IC₅₀ values | Observed | synergism |
| Sample | [A] | [B] | [A + B]/2 | IC₅₀ values | [exp./obs] |
| Ascorbic acid | 2.10 ± 0.25 | 4.92 ± 0.26 | 3.51 | 2.64 ± 0.58 | 1.33 |
| preparation + | | | | | |
| 40% Pomegranate | | | | | |
| in 1:1 w/w ratio | | | | | |
| Ascorbic acid | 2.10 ± 0.25 | 10.31 ± 0.88 | 6.21 | 3.63 ± 0.56 | 1.71 |
| preparation + | | | | | |
| 20% Pomegranate | | | | | |
| in 1:1 w/w ratio | | | | | |
| Ascorbic acid | 2.10 ± 0.25 | 18.38 ± 0.66 | 10.24 | 4.09 ± 0.63 | 2.50 |
| preparation + | | | | | |
| 10% Pomegranate | | | | | |
| in 1:1 w/w ratio | | | | | |

TABLE 7
Degree of synergism of mixtures of Ascorbic acid preparation and three pomegranate
extracts in DPPH radical cation decolorisation assay

| | IC₅₀ values of individual | | | | |
|---|---|---|---|---|---|
| | components | | | | |
| | Ascorbic | | | | |
| | acid | Pomegranate | Expected | | Degree of |
| | preparation | Sample | IC₅₀ values | Observed | synergism |
| Sample | [A] | [B] | [A + B]/2 | IC₅₀ values | [exp./obs] |
| Ascorbic acid | 6.91 ± 0.83 | 12.19 ± 0.79 | 9.55 | 6.98 ± 0.74 | 1.37 |
| preparation + | | | | | |
| 40% Pomegranate | | | | | |
| in 1:1 w/w ratio | | | | | |
| Ascorbic acid | 6.91 ± 0.83 | 19.43 ± 0.88 | 13.17 | 8.92 ± 0.75 | 1.48 |
| preparation + | | | | | |
| 20% Pomegranate | | | | | |
| in 1:1 w/w ratio | | | | | |
| Ascorbic acid | 6.91 ± 0.83 | 35.72 ± 2.20 | 21.31 | 12.11 ± 1.22 | 1.76 |
| preparation + | | | | | |
| 10% Pomegranate | | | | | |
| in 1:1 w/w ratio | | | | | |

TABLE 8
Degree of synergism of mixtures of Antioxidant Composition and three
pomegranate extracts in ABTS radical cation decolorisation assay

| | IC₅₀ values | | | | |
|---|---|---|---|---|---|
| | of individual | | | | |
| | components | | | | |
| | Antioxidant | Pomegranate | Expected | | Degree of |
| | Composition | Sample | IC₅₀ values | Observed | synergism |
| Sample | [A] | [B] | [A + B]/2 | IC₅₀ values | [exp./obs] |
| Antioxidant | 2.47 ± 0.36 | 4.92 ± 0.26 | 3.70 | 2.57 ± 0.43 | 1.43 |
| Composition + | | | | | |
| 40% Pomegranate | | | | | |
| in 1:1 w/w ratio | | | | | |
| Antioxidant | 2.47 ± 0.36 | 10.31 ± 0.88 | 6.39 | 3.75 ± 0.82 | 1.70 |
| Composition + | | | | | |
| 20% Pomegranate | | | | | |
| in 1:1 w/w ratio | | | | | |
| Antioxidant | 2.47 ± 0.36 | 18.38 ± 0.66 | 10.43 | 4.48 ± 0.45 | 2.33 |
| Composition + | | | | | |
| 10% Pomegranate | | | | | |
| in 1:1 w/w ratio | | | | | |

TABLE 9
Degree of synergism of mixtures of Antioxidant Composition and three pomegranate
extracts in DPPH radical cation decolorisation assay

| | IC₅₀ values of individual | | | | |
|---|---|---|---|---|---|
| | components | | | | |
| | Antioxidant | Pomegranate | Expected | | Degree of |
| | Composition | Sample | IC₅₀ values | Observed | synergism |
| Sample | [A] | [B] | [A + B]/2 | IC₅₀ values | [exp./obs] |
| Antioxidant | 8.67 ± 0.22 | 12.19 ± 0.79 | 10.43 | 9.02 ± 0.42 | 1.16 |
| Composition + | | | | | |
| 40% Pomegranate | | | | | |
| in 1:1 w/w ratio | | | | | |
| Antioxidant | 8.67 ± 0.22 | 19.43 ± 0.88 | 14.05 | 11.52 ± 0.72 | 1.22 |
| Composition + | | | | | |
| 20% Pomegranate | | | | | |
| in 1:1 w/w ratio | | | | | |
| Antioxidant | 8.67 ± 0.22 | 35.72 ± 2.20 | 22.20 | 15.72 ± 1.09 | 1.41 |
| Composition + | | | | | |
| 10% Pomegranate | | | | | |
| in 1:1 w/w ratio | | | | | |

### Experimental Example 5

An extract of pomegranate (having 40% bioactives) was combined with dextrin white in ratios of 1:7 weight to weight (w/w), 1:15 (w/w), and 1:39 (w/w) to achieve samples with bioactive concentrations of 5%, 2.5% and 1%, respectively.

An ascorbic acid preparation was prepared according to Example 1 above. An extract of Indian gooseberry was prepared according to Example 1 above. An antioxidant composition was prepared by a combination of 125 mg of the ascorbic acid preparation to 75 mg of the extract of Indian gooseberry (5:3 w/w).

The antioxidant effect of each of the extract of Indian gooseberry, ascorbic acid preparation, and antioxidant composition with each of the extracts of pomegranate was tested. Each of the extract of Indian gooseberry, ascorbic acid preparation, and the antioxidant composition were combined separately with samples of the three pomegranate extracts by dissolving in distilled water separately and mixing to obtain a final concentration of 1 mg/ml of the mixture containing the two components in a 1:1 w/w ratio. Individual components and the combined mixtures were assayed for their antioxidant profiles in vitro.

Synergism was calculated from the following formula—

$$Degree of synergism = \frac{Expected {IC}_{50} of the mixture}{Observed {IC}_{50} of the mixture}$$

$$Expected {IC}_{50} of the mixture = Mean of the {IC}_{50} values of the individual components$$

Since, in case of synergism, the observed antioxidant profile in terms of IC₅₀ of the mixture would be lower than the expected IC₅₀ value, the degree of synergism would be greater than 1. Higher values denote profound synergism.

ABTS and DPPH radical decolorisation assays of the individual samples were performed and the antioxidant profile has been provided for each in Table 10 and 11. Synergism data is provided for the extract of Indian gooseberry, ascorbic acid preparation, and antioxidant composition each with each of the pomegranate extracts below in Tables 12 to 17. A considerable synergism was observed between the antioxidant composition and each of the three pomegranate extracts. See Tables 16 and 17. The ABTS radical cation decolorisation assay was performed according to the method of Pellegrini et al. (2003) (see above). Briefly, neutralization of the radical and reduction of color at 734 nm by the antioxidants was measured and calculated with respect to the control. The results were expressed as IC₅₀ of the samples. The DPPH radical decolorisation assay was performed according to the method of Gulcin et al. (2004) (see above). Briefly, neutralization of the radical and reduction of color at 517 nm by the antioxidants was measured and calculated with respect to the control. The results were expressed as IC₅₀ of the samples.

TABLE 10
IC₅₀ values (in micrograms/ml) of different extracts
in ABTS radical cation decolorisation assay

| Sample | IC₅₀ values |
|---|---|
| Antioxidant Composition | 2.47 ± 0.36 |
| Extract of Indian Gooseberry | 3.03 ± 0.19 |
| Ascorbic Acid Preparation | 2.10 ± 0.25 |
| 5% Pomegranate | 26.76 ± 4.15 |
| 2.5% Pomegranate | 47.42 ± 4.85 |
| 1% Pomegranate | 71.99 ± 5.19 |

TABLE 11
IC₅₀ values (in micrograms/ml) of different
extracts in DPPH radical decolorisation assay

| Sample | IC₅₀ values |
|---|---|
| Antioxidant Composition | 8.67 ± 0.22 |
| Extract of Indian Gooseberry | 10.77 ± 0.86 |
| Ascorbic Acid Preparation | 6.91 ± 0.57 |
| 5% Pomegranate | 46.70 ± 1.98 |
| 2.5% Pomegranate | 81.86 ± 7.04 |
| 1% Pomegranate | 202.43 ± 25.01 |

TABLE 12
Degree of synergism of mixtures of Extract of Indian gooseberry and three
pomegranate extracts in ABTS radical cation decolorisation assay

| | IC₅₀ values of individual | | | | |
|---|---|---|---|---|---|
| | components | | | | |
| | Extract of | | | | |
| | Indian | Other | Expected | | Degree of |
| | gooseberry | antioxidant | IC₅₀ values | Observed | synergism |
| Sample | [A] | [B] | [A + B]/2 | IC₅₀ values | [exp./obs] |
| Extract of Indian | 3.03 ± 0.19 | 26.76 ± 4.15 | 14.90 | 7.08 ± 1.13 | 2.10 |
| gooseberry + 5% | | | | | |
| Pomegranate in | | | | | |
| 1:1 w/w ratio | | | | | |
| Extract of Indian | 3.03 ± 0.19 | 47.42 ± 4.85 | 25.22 | 8.47 ± 0.63 | 2.98 |
| gooseberry + | | | | | |
| 2.5% | | | | | |
| Pomegranate in | | | | | |
| 1:1 w/w ratio | | | | | |
| Extract of Indian | 3.03 ± 0.19 | 71.99 ± 5.19 | 37.51 | 9.41 ± 0.82 | 3.99 |
| gooseberry + 1% | | | | | |
| Pomegranate in | | | | | |
| 1:1 w/w ratio | | | | | |

TABLE 13
Degree of synergism of mixtures of Extract of Indian gooseberry and three
pomegranate extracts in DPPH radical cation decolorisation assay

| | IC₅₀ values of individual | | | | |
|---|---|---|---|---|---|
| | components | | | | |
| | Extract of | | | | |
| | Indian | Other | Expected | | Degree of |
| | gooseberry | antioxidant | IC₅₀ values | Observed | synergism |
| Sample | [A] | [B] | [A + B]/2 | IC₅₀ values | [exp./obs] |
| Extract of Indian | 10.77 ± 0.86 | 46.70 ± 1.98 | 28.73 | 19.90 ± 0.66 | 1.44 |
| gooseberry + 5% | | | | | |
| Pomegranate in | | | | | |
| 1:1 w/w ratio | | | | | |
| Extract of Indian | 10.77 ± 0.86 | 81.86 ± 7.04 | 46.31 | 22.98 ± 5.55 | 2.02 |
| gooseberry + | | | | | |
| 2.5% | | | | | |
| Pomegranate in | | | | | |
| 1:1 w/w ratio | | | | | |
| Extract of Indian | 10.77 ± 0.86 | 202.43 ± 25.01 | 106.60 | 39.30 ± 6.58 | 2.71 |
| gooseberry + 1% | | | | | |
| Pomegranate in | | | | | |
| 1:1 w/w ratio | | | | | |

TABLE 14
Degree of synergism of mixtures of Ascorbic acid preparation and three
pomegranate extracts in ABTS radical cation decolorisation assay

| | IC₅₀ values of individual | | | | |
|---|---|---|---|---|---|
| | components | | | | |
| | Ascorbic | | | | |
| | acid | Other | Expected | | Degree of |
| | preparation | antioxidant | IC₅₀ values | Observed | synergism |
| Sample | [A] | [B] | [A + B]/2 | IC₅₀ values | [exp./obs] |
| Ascorbic acid | 2.10 ± 0.25 | 26.76 ± 4.15 | 14.43 | 4.75 ± 0.33 | 3.04 |
| preparation + 5% | | | | | |
| Pomegranate in | | | | | |
| 1:1 w/w ratio | | | | | |
| Ascorbic acid | 2.10 ± 0.25 | 47.42 ± 4.85 | 24.76 | 6.60 ± 0.61 | 3.75 |
| preparation + | | | | | |
| 2.5% | | | | | |
| Pomegranate in | | | | | |
| 1:1 w/w ratio | | | | | |
| Ascorbic acid | 2.10 ± 0.25 | 71.99 ± 5.19 | 37.05 | 8.27 ± 0.81 | 4.48 |
| preparation + 1% | | | | | |
| Pomegranate in | | | | | |
| 1:1 w/w ratio | | | | | |

TABLE 15
Degree of synergism of mixtures of Ascorbic acid preparation and three
pomegranate extracts in DPPH radical cation decolorisation assay

| | IC₅₀ values of individual | | | | |
|---|---|---|---|---|---|
| | components | | | | |
| | Ascorbic | | | | |
| | acid | Other | Expected | | Degree of |
| | preparation | antioxidant | IC₅₀ values | Observed | synergism |
| Sample | [A] | [B] | [A + B]/2 | IC₅₀ values | [exp./obs] |
| Ascorbic acid | 6.91 ± 0.83 | 46.70 ± 1.98 | 26.81 | 14.15 ± 2.02 | 1.89 |
| preparation + 5% | | | | | |
| Pomegranate in | | | | | |
| 1:1 w/w ratio | | | | | |
| Ascorbic acid | 6.91 ± 0.83 | 81.86 ± 7.04 | 44.39 | 18.37 ± 1.60 | 2.42 |
| preparation + | | | | | |
| 2.5% | | | | | |
| Pomegranate in | | | | | |
| 1:1 w/w ratio | | | | | |
| Ascorbic acid | 6.91 ± 0.83 | 202.43 ± 25.01 | 104.67 | 25.90 ± 0.28 | 4.04 |
| preparation + 1% | | | | | |
| Pomegranate in | | | | | |
| 1:1 w/w ratio | | | | | |

TABLE 16
Degree of synergism of mixtures of Antioxidant Composition and three
pomegranate extracts in ABTS radical cation decolorisation assay

| | IC₅₀ values of individual | | | | |
|---|---|---|---|---|---|
| | components | | | | |
| | Antioxidant | Other | Expected | | Degree of |
| | Composition | antioxidant | IC₅₀ values | Observed | synergism |
| Sample | [A] | [B] | [A + B]/2 | IC₅₀ values | [exp./obs] |
| Antioxidant | 2.47 ± 0.36 | 26.76 ± 4.15 | 14.61 | 5.07 ± 0.58 | 2.88 |
| Composition + | | | | | |
| 5% Pomegranate | | | | | |
| in 1:1 w/w ratio | | | | | |
| Antioxidant | 2.47 ± 0.36 | 47.42 ± 4.85 | 24.94 | 6.57 ± 0.78 | 3.79 |
| Composition + | | | | | |
| 2.5% | | | | | |
| Pomegranate in | | | | | |
| 1:1 w/w ratio | | | | | |
| Antioxidant | 2.47 ± 0.36 | 71.99 ± 5.19 | 37.23 | 8.23 ± 0.69 | 4.52 |
| Composition + | | | | | |
| 1% Pomegranate | | | | | |
| in 1:1 w/w ratio | | | | | |

TABLE 17
Degree of synergism of mixtures of Antioxidant Composition and three
pomegranate extracts in DPPH radical cation decolorisation assay

| | IC₅₀ values of individual | | | | |
|---|---|---|---|---|---|
| | components | | | | |
| | Antioxidant | Other | Expected | | Degree of |
| | Composition | antioxidant | IC₅₀ values | Observed | synergism |
| Sample | [A] | [B] | [A + B]/2 | IC₅₀ values | [exp./obs] |
| Antioxidant | 8.67 ± 0.22 | 46.70 ± 1.98 | 27.68 | 17.64 ± 0.95 | 1.57 |
| Composition + | | | | | |
| 5% Pomegranate | | | | | |
| in 1:1 w/w ratio | | | | | |
| Antioxidant | 8.67 ± 0.22 | 81.86 ± 7.04 | 45.27 | 21.93 ± 1.50 | 2.06 |
| Composition + | | | | | |
| 2.5% | | | | | |
| Pomegranate in | | | | | |
| 1:1 w/w ratio | | | | | |
| Antioxidant | 8.67 ± 0.22 | 202.43 ± 25.01 | 102.45 | 34.94 ± 3.25 | 2.93 |
| Composition + | | | | | |
| 1% Pomegranate | | | | | |
| in 1:1 w/w ratio | | | | | |

### Experimental Example 6

A sample of each of five extracts of the superfruits (pomegranate, blueberry, cranberry, goji, and acai) was prepared. The pomegranate extract (standardized to 40% ellagic acid) was a brown solid available from Novel Ingredients Services, LLC of Los Angeles, Calif. The blueberry extract (25:1) was a dark magenta solid available from Draco Natural Products Inc. of San Jose, Calif. The cranberry extract (10:1) was a rose-red powder available from Draco Natural Products, Inc. The acai extract (4:1) was a brown solid available from Nature's Pure Nutraceuticals Corp. (NP Nutra) of Gardena, Calif. The goji extract (standardized to 20% polysaccharides) was a golden yellow solid available from NP Nutra.

An ascorbic acid preparation was prepared according to Example 1 above. An extract of Indian gooseberry was prepared according to Example 1 above. An antioxidant composition was prepared by a combination of 125 mg of the ascorbic acid preparation to 75 mg of the extract of Indian gooseberry (5:3 w/w).

The antioxidant effect of the extract of Indian gooseberry with each of the superfruit extract samples and of the antioxidant composition with each of the superfruit extract samples was tested. Each of the extract of Indian gooseberry and the antioxidant composition were combined separately with each of the five superfruit extracts by dissolving each combination in distilled water separately and mixing to obtain a final concentration of 1 mg/ml of the mixture containing the two components in 1:1 w/w ratio. Individual components and the synergistic mixtures were assayed for their antioxidant profiles in vitro.

Synergism was calculated from the following formula—

$$Degree of synergism = \frac{Expected {IC}_{50} of the mixture}{Observed {IC}_{50} of the mixture}$$

$$Expected {IC}_{50} of the mixture = Mean of the {IC}_{50} values of the individual components$$

Since, in case of synergism, the observed antioxidant profile in terms of IC₅₀ of the mixture would be lower than the expected IC₅₀ value, the degree of synergism would be greater than 1. Higher values denote profound synergism.

ABTS and DPPH radical decolorisation assays of the individual samples were performed and the antioxidant profile has been provided for each in Tables 21 and 23. Synergism data is provided for the extract of Indian gooseberry and antioxidant composition each with each of the superfruit extract samples below in Tables 19 to 20, 22, and 24. A considerable synergism was observed between the antioxidant composition and each of the five superfruit extracts. See Tables 22 and 24. The ABTS radical cation decolorisation assay was performed according to the method of Pellegrini et al. (2003). (see above). Briefly, neutralization of the radical and reduction of color at 734 nm by the antioxidants was measured and calculated with respect to the control. The results were expressed as IC₅₀ of the samples. The DPPH radical decolorisation assay was performed according to the method of Gulcin et al. (2004). (see above). Briefly, neutralization of the radical and reduction of color at 517 nm by the antioxidants was measured and calculated with respect to the control. The results were expressed as IC₅₀ of the samples.

TABLE 19
IC₅₀ values (in μg/ml) and degree of synergism of mixtures of
Extract of Indian gooseberry and five super fruit extracts in ABTS radical
cation decolorisation assay

| | IC₅₀ values of individual | | | | |
|---|---|---|---|---|---|
| | components | | | | |
| | Extract of | | | | |
| | Indian | Other | Expected | | Degree of |
| | gooseberry | antioxidants | IC₅₀ values | Observed | synergism |
| Sample | [A] | [B] | [A + B]/2 | IC₅₀ values | [exp./obs] |
| Extract of Indian | 3.03 ± 0.19 | 4.92 ± 0.26 | 3.98 | 3.19 ± 0.64 | 1.25 |
| gooseberry + | | | | | |
| Pomegranate in | | | | | |
| 1:1 w/w ratio | | | | | |
| Extract of Indian | 3.03 ± 0.19 | 63.22 ± 2.61 | 33.13 | 6.98 ± 0.75 | 4.75 |
| gooseberry + | | | | | |
| Blueberry in 1:1 | | | | | |
| w/w ratio | | | | | |
| Extract of Indian | 3.03 ± 0.19 | 66.18 ± 1.78 | 34.61 | 7.91 ± 0.59 | 4.38 |
| gooseberry + | | | | | |
| Cranberry in 1:1 | | | | | |
| w/w ratio | | | | | |
| Extract of Indian | 3.03 ± 0.19 | 103.60 ± 9.89 | 53.32 | 9.02 ± 0.32 | 5.91 |
| gooseberry + | | | | | |
| Acai in 1:1 w/w | | | | | |
| ratio | | | | | |
| Extract of Indian | 3.03 ± 0.19 | 81.02 ± 3.91 | 42.03 | 8.83 ± 0.44 | 4.76 |
| gooseberry + Goji | | | | | |
| in 1:1 w/w ratio | | | | | |

TABLE 20
IC₅₀ values (in μg/ml) and degree of synergism of mixtures of
Extract of Indian gooseberry and five super fruit extracts in DPPH radical
cation decolorisation assay

| | IC₅₀ values of individual | | | | |
|---|---|---|---|---|---|
| | components | | | | |
| | Extract of | | Expected | | |
| | Indian | Other | IC₅₀ | | Degree of |
| | gooseberry | antioxidants | values | Observed | synergism |
| Sample | [A] | [B] | [A + B]/2 | IC₅₀ values | [exp./obs] |
| Extract of Indian | 10.77 ± 0.86 | 12.19 ± 0.79 | 11.48 | 10.41 ± 0.41 | 1.10 |
| gooseberry + | | | | | |
| Pomegranate in | | | | | |
| 1:1 w/w ratio | | | | | |
| Extract of Indian | 10.77 ± 0.86 | 617.36 ± 11.31 | 314.07 | 19.74 ± 0.85 | 15.91 |
| gooseberry + | | | | | |
| Blueberry in 1:1 | | | | | |
| w/w ratio | | | | | |
| Extract of Indian | 10.77 ± 0.86 | 726.29 ± 22.86 | 368.53 | 20.12 ± 0.61 | 18.32 |
| gooseberry + | | | | | |
| Cranberry in 1:1 | | | | | |
| w/w ratio | | | | | |
| Extract of Indian | 10.77 ± 0.86 | 2031.49 ± 174.82 | 1021.13 | 22.23 ± 0.86 | 45.93 |
| gooseberry + | | | | | |
| Acai in 1:1 w/w | | | | | |
| ratio | | | | | |
| Extract of Indian | 10.77 ± 0.86 | 953.33 ± 83.78 | 482.39 | 22.53 ± 0.93 | 21.41 |
| gooseberry + Goji | | | | | |
| in 1:1 w/w ratio | | | | | |

TABLE 21
IC₅₀ values (in μg/ml) of Antioxidant Composition
and five super fruit extracts in ABTS radical
cation decolorisation assay

| Sample | IC₅₀ values |
|---|---|
| Antioxidant Composition | 2.47 ± 0.36 |
| Pomegranate | 4.92 ± 0.26 |
| Blueberry | 63.22 ± 2.61 |
| Cranberry | 66.18 ± 1.78 |
| Acai | 103.60 ± 9.89 |
| Goji | 81.02 ± 3.91 |

TABLE 22
IC₅₀ values (in μg/ml) and degree of synergism of mixtures of
Antioxidant Composition and five super fruit extracts in ABTS radical
cation decolorisation assay

| | IC₅₀ values of individual | | | | |
|---|---|---|---|---|---|
| | components | | | | |
| | Antioxidant | Other | Expected | | Degree of |
| | Composition | antioxidants | IC₅₀ values | Observed | synergism |
| Sample | [A] | [B] | [A + B]/2 | IC₅₀ values | [exp./obs] |
| Antioxidant | 2.47 ± 0.36 | 4.92 ± 0.26 | 3.70 | 2.58 ± 0.43 | 1.43 |
| Composition + | | | | | |
| Pomegranate in | | | | | |
| 1:1 w/w ratio | | | | | |
| Antioxidant | 2.47 ± 0.36 | 63.22 ± 2.61 | 32.84 | 5.68 ± 0.66 | 5.78 |
| Composition + | | | | | |
| Blueberry in 1:1 | | | | | |
| w/w ratio | | | | | |
| Antioxidant | 2.47 ± 0.36 | 66.18 ± 1.78 | 34.32 | 5.61 ± 0.44 | 6.12 |
| Composition + | | | | | |
| Cranberry in 1:1 | | | | | |
| w/w ratio | | | | | |
| Antioxidant | 2.47 ± 0.36 | 103.60 ± 9.89 | 53.04 | 6.25 ± 0.31 | 8.49 |
| Composition + | | | | | |
| Acai in 1:1 w/w | | | | | |
| ratio | | | | | |
| Antioxidant | 2.47 ± 0.36 | 81.02 ± 3.91 | 41.75 | 5.93 ± 0.31 | 7.04 |
| Composition + | | | | | |
| Goji in 1:1 w/w | | | | | |
| ratio | | | | | |

TABLE 23
IC₅₀ values (in μg/ml) of Antioxidant Composition
and five super fruit extracts in DPPH radical
decolorisation assay

| Sample | IC₅₀ values |
|---|---|
| Antioxidant Composition | 8.67 ± 0.22 |
| Pomegranate | 12.19 ± 0.79 |
| Blueberry | 617.36 ± 11.31 |
| Cranberry | 726.29 ± 22.86 |
| Acai | 2031.49 ± 174.82 |
| Goji | 953.33 ± 83.78 |

TABLE 24
IC₅₀ values (in μg/ml) and degree of synergism of mixtures of
Antioxidant Composition and five super fruit extracts in DPPH radical
decolorisation assay

| | IC₅₀ values of individual | | | | |
|---|---|---|---|---|---|
| | components | | Expected | | |
| | Antioxidant | Other | IC₅₀ | | Degree of |
| | Composition | antioxidants | values | Observed | synergism |
| Sample | [A] | [B] | [A + B]/2 | IC₅₀ values | [exp./obs] |
| Antioxidant | 8.67 ± 0.22 | 12.19 ± 0.79 | 10.43 | 9.02 ± 0.42 | 1.16 |
| Composition + | | | | | |
| Pomegranate in | | | | | |
| 1:1 w/w ratio | | | | | |
| Antioxidant | 8.67 ± 0.22 | 617.36 ± 11.31 | 313.01 | 18.71 ± 0.84 | 16.72 |
| Composition + | | | | | |
| Blueberry in 1:1 | | | | | |
| w/w ratio | | | | | |
| Antioxidant | 8.67 ± 0.22 | 726.29 ± 22.86 | 367.48 | 18.56 ± 0.47 | 19.80 |
| Composition + | | | | | |
| Cranberry in 1:1 | | | | | |
| w/w ratio | | | | | |
| Antioxidant | 8.67 ± 0.22 | 2031.49 ± 174.82 | 1020.08 | 19.96 ± 0.57 | 51.11 |
| Composition + | | | | | |
| Acai in 1:1 w/w | | | | | |
| ratio | | | | | |
| Antioxidant | 8.67 ± 0.22 | 953.33 ± 83.78 | 481.00 | 19.31 ± 0.32 | 24.91 |
| Composition + | | | | | |
| Goji in 1:1 w/w | | | | | |
| ratio | | | | | |

TABLE 25
IC₅₀ values (in μg/ml) and degree of synergism of mixtures of
extract of Indian gooseberry and ascorbic acid preparation in an exemplary
Antioxidant Composition according to the present disclosure in ABTS radical
cation decolorisation assay

| | IC₅₀ values of individual | | | | |
|---|---|---|---|---|---|
| | components | | | | |
| | extract of | ascorbic acid | Expected | | Degree |
| | Indian | preparation | IC₅₀ values | Observed | of synergism |
| Sample | gooseberry [A] | [B] | [A + B]/2 | IC₅₀ values | [exp./obs] |
| Antioxidant | 3.03 ± 0.19 | 2.10 ± 0.14 | 2.57 | 2.47 ± 0.36 | 1.04 |
| Composition | | | | | |

TABLE 26
IC₅₀ values (in μg/ml) and degree of synergism of mixtures of
extract of Indian gooseberry and ascorbic acid preparation in an exemplary
Antioxidant Composition according to the present disclosure in DPPH
radical decolorisation assay

| | IC₅₀ values of individual | | | | |
|---|---|---|---|---|---|
| | components | | | | |
| | extract of | ascorbic acid | Expected | | Degree |
| | Indian | preparation | IC₅₀ values | Observed | of synergism |
| Sample | gooseberry [A] | [B] | [A + B]/2 | IC₅₀ values | [exp./obs] |
| Antioxidant | 10.77 ± 0.86 | 6.91 ± 0.57 | 8.84 | 8.67 ± 0.22 | 1.02 |
| Composition | | | | | |

Referring to Experimental Examples 4 and 5 the greatest synergism occurred between the antioxidant composition and the 1% pomegranate extract while the least occurred between the antioxidant composition and the 40% pomegranate extract. Although the exact reasons for this result are not known, it is possible that the hydrolyzable tannin bioactives in the pomegranate extract are similar to those of the antioxidant composition. In one exemplary aspect, the level of antioxidant synergism may be related to the amount of bioactives and subsequent activity level of antioxidants in the superfruit being combined with the antioxidant composition. Referring to Experimental Example 6, differences in bioactive constituents appear to influence the amount of synergism to some degree. The 1% pomegranate extract had an ABTS IC₅₀ level of 71.99 while the cranberry extract had a comparable IC₅₀ level of 66.18. Synergism between the antioxidant composition and 1% pomegranate extract was 4.52 in the ABTS assay while synergism between the antioxidant composition and cranberry extract was 6.12. It is also observed that the relationship between the absolute synergism values for the ABTS and DPPH assays was different for pomegranate extract than for the other superfruit extracts tested. For pomegranate extract, the ABTS assay resulted in greater synergism values than the DPPH assay, which is opposite of what was observed for the other superfruit extracts. Although the exact reasons for this result are not known, it is possible that this could be related to a difference in how the bioactives of each superfruit interact with the antioxidant composition and the indicator chemical in each assay.

It is also noted that Example 6 shows little to no degree of synergism in combining the exemplary extract of Indian gooseberry with the exemplary ascorbic acid preparation (see Tables 25 and 26).

Example synergistically increased antioxidant compositions are contemplated that result in at least the degree of synergism set forth in the “degree of synergism” columns of the tables set forth above with respect to Experimental Examples 4 to 6. Example synergistically increased antioxidant compositions are also contemplated that result in the degree of synergism set forth in the “degree of synergism” columns of the tables set forth above with respect to Experimental Examples 4 to 6.

### Experimental Example 7

An ascorbic acid preparation was prepared according to Example 1 above. An extract of Indian gooseberry was prepared according to Example 1 above. An antioxidant composition was prepared by a combination of 125 mg of the ascorbic acid preparation to 75 mg of the extract of Indian gooseberry (5:3 w/w).

The antioxidant composition was combined with a variety of juices and green tea to achieve 8 ounce (oz.) samples for testing. The juices/teas examined were commercially available cranberry juice, apple juice (filtered), natural apple juice (unfiltered), green tea, tomato juice, and orange juice. For each juice and green tea, a sample of 8 oz. of the juice/tea was prepared. Additionally, combinations of 100 mg of the antioxidant composition and each juice/tea were prepared to achieve 8 oz. samples. Further, combinations of 200 mg of the antioxidant composition and each juice/tea were prepared to achieve an 8 oz. sample. Each of the samples was tested using an ABTS radical cation decolorization assay performed according to the method of Miller, N. et al. (Miller, N. and Rice Evans, C. Factors influencing the antioxidant activity determined by ABTS radical cation assay. Free Rad Res. 26, 195-199 (1997), which is incorporated herein by reference in its entirety.). The results were expressed as millimolar (mM) Trolox equivalents (TE) ABTS values. Table 27 illustrates mM TE results for each sample and a calculation of the percentage increase of antioxidant effect with the addition either 100 mg or 200 mg of antioxidant composition. Table 28 illustrates the synergism achieved by the combinations with the 200 mg samples using the synergism procedures outlined above. An 8 oz. sample including 200 mg of antioxidant composition in water was also prepared and an ABTS assay was obtained. The ABTS value for this sample was 2.43 mM TE.

TABLE 27
ABTS Assay Data for Various Juice/Tea and
Antioxidant Composition Samples

| | | | % |
|---|---|---|---|
| Beverage Type | Sample Tested (8 oz.) | mM TE | Increase |
| Cranberry Juice | Juice Only | 0.7 | |
| Cranberry Juice | Juice plus 100 mg Antioxidant | 1.9 | 179.8 |
| | Composition | | |
| Cranberry Juice | Juice plus 200 mg Antioxidant | 2.3 | 243.4 |
| | Composition | | |
| Apple Juice | Juice Only | 0.1 | |
| Apple Juice | Juice plus 100 mg Antioxidant | 1.2 | 2107.9 |
| | Composition | | |
| Apple Juice | Juice plus 200 mg Antioxidant | 2.5 | 4423.8 |
| | Composition | | |
| Natural Apple | Juice Only | 0.7 | |
| Juice | | | |
| Natural Apple | Juice plus 100 mg Antioxidant | 1.4 | 110.0 |
| Juice | Composition | | |
| Natural Apple | Juice plus 200 mg Antioxidant | 2.8 | 322.1 |
| Juice | Composition | | |
| Green Tea | Tea Only | 1.9 | |
| Green Tea | Tea plus 100 mg Antioxidant | 3.6 | 93.3 |
| | Composition | | |
| Green Tea | Tea plus 200 mg Antioxidant | 4.4 | 139.5 |
| | Composition | | |
| Tomato Juice | Juice Only | 1.7 | |
| Tomato Juice | Juice plus 100 mg Antioxidant | 3.1 | 89.2 |
| | Composition | | |
| Tomato Juice | Juice plus 200 mg Antioxidant | 4.5 | 170.1 |
| | Composition | | |
| Orange Juice | Juice Only | 0.21 | |
| Orange Juice | Juice plus 100 mg Antioxidant | 1.06 | 393.4 |
| | Composition | | |
| Orange Juice | Juice plus 200 mg Antioxidant | 1.94 | 804.3 |
| | Composition | | |

TABLE 28
Synergism Data for Juice/Tea Combinations with the Exemplary Antioxidant
Composition

| | mM | | | | |
|---|---|---|---|---|---|
| | TE 8 oz. | mM TE for 200 mg | Expected | Observed | |
| | juice | antioxidant | mM | mM TE | |
| | or tea | composition in | TE of the | of the | |
| Beverage | only | 8 oz. water | combination | combination | Synergism |
| Cranberry Juice | 0.7 | 2.43 | 1.565 | 1.8 | 1.2 |
| Apple Juice | 0.1 | 2.43 | 1.265 | 1.3 | 1.0 |
| Natural Apple | 0.7 | 2.43 | 1.565 | 0.9 | 0.6 |
| Juice | | | | | |
| Green tea | 1.9 | 2.43 | 2.165 | 2.7 | 1.2 |
| Tomato juice | 1.7 | 2.43 | 2.065 | 2.3 | 1.1 |
| Orange juice | 0 | 2.43 | 1.215 | 0.8 | 0.7 |

Example synergistically increased antioxidant compositions are contemplated that result in at least the degree of synergism set forth in the “degree of synergism” column of Table 28 set forth above. Example synergistically increased antioxidant compositions are also contemplated that result in the degree of synergism set forth in the “degree of synergism” columns of Table 28 set forth above. Other example synergistically increased compositions are contemplated that result in at least a percent increase in ABTS antioxidant activity as set forth in Table 27 above. Additional example synergistically increased compositions are contemplated that result in a percent increase in ABTS antioxidant activity as set forth in Table 27 above.

### Experimental Example 8

An ascorbic acid preparation was prepared according to Example 1 above. An extract of Indian gooseberry was prepared according to Example 1 above. Various combinations of the ascorbic acid preparation and the Indian gooseberry preparation were combined with different juices and antioxidant capacity in mM TE were determined. A TEAC Antioxidant Assay Kit (Catalog no. 709001, Cayman Chemical Co., Ann Arbor, Mich.) was used. Prior to antioxidant capacity testing, samples were prepared as follows. Eight ounce samples of each of commercially sold 100% apple juice (fortified with 100% of the RDA for Vitamin C; Sample 1) and unfortified 100% apple juice (Sample 2) were tested as is. Various amounts or combinations of the ascorbic acid preparation and the Indian gooseberry preparation (as set forth in Table 29) were added to 8 oz. unfortified 100% apple juice to produce Samples 3-5. Sample 1 was tested for antioxidant capacity without any further pasteurization since it had been pasteurized during the commercial bottling process. Prior to antioxidant testing, Samples 2-5 were pasteurized using a benchtop pasteurization procedure.

Antioxidant capacities for each sample appear in Table 29. Unfortified 100% apple juice had no antioxidant capacity while commercially sold 100% apple juice was 0.090 mM TE. The combination of the ascorbic acid preparation and the Indian gooseberry preparation enhanced the antioxidant capacity of the unfortified 100% apple juice by between 486-1917%, depending upon the amount of the two preparations added and the ratio of the two preparations. These results demonstrate the ability of the synergistic combinations of the two preparations with the juice to greatly increase the antioxidant level of low antioxidant juices such as apple juice, producing a beverage that has superior antioxidant capacity when compared to commercially sold 100% apple juice.

TABLE 29
Antioxidant capacities of commercially sold
100% apple juice, unfortified 100% apple juice,
and unfortified 100% apple juice to which an ascorbic
acid preparation and an Indian gooseberry preparation
were added in various dosages and ratios.

| | | Antioxidant | |
|---|---|---|---|
| Sample | | capacity | Percent |
| number | Sample identity | (mM TE) | increase |
| 1 | Commercially sold 100% apple juice | 0.090 | |
| 2 | Unfortified 100% apple juice | 0.000 | |
| 3 | Unfortified 100% apple juice + 100 mg | 0.486 | 486% |
| | of a combination of 62.5 mg of the | | |
| | ascorbic acid preparation and 37.5 mg | | |
| | of the Indian Gooseberry preparation) | | |
| 4 | Unfortified 100% apple juice + 200 mg | 1.917 | 1917% |
| | of a combination of 125 mg of the | | |
| | ascorbic acid preparation and 75 mg | | |
| | of the Indian Gooseberry extract | | |
| 5 | Unfortified 100% apple juice + 160 mg | 1.715 | 1715% |
| | of a combination of 122.5 mg of the | | |
| | ascorbic acid preparation and 37.5 mg | | |
| | Indian Gooseberry extract | | |

It is contemplated that in any one or more of the examples included herein, that one or more of the constituents listed in an amount qualified by the term “about” may be present in an amount that is not qualified by the term “about” and for examples not so qualified that other examples that are qualified with the term “about” are also contemplated. For the sake of efficiency only, such additional examples are not listed further herein.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A composition having increased antioxidant capability, the composition comprising a synergistically effective combination of:
an Indian gooseberry extract;
ascorbic acid; and
a target component,
wherein the Indian gooseberry extract, the ascorbic acid, and the target component are present in amounts effective to synergistically increase the antioxidant capability of the target component.

2. A composition according to claim 1, wherein the composition has an ABTS degree of synergism of at least about 1.2.

3. A composition according to claim 1, wherein the composition has an ABTS degree of synergism of at least about 1.7.

4. A composition according to claim 1, wherein the composition has an ABTS degree of synergism of at least about 2.

5. A composition according to claim 1, wherein the composition has an ABTS degree of synergism of at least about 5.

6. A composition according to claim 1, wherein the composition has a DPPH degree of synergism of at least about 1.1.

7. A composition according to claim 1, wherein the composition has a DPPH degree of synergism of at least about 2.

8. A composition according to claim 1, wherein the composition has a DPPH degree of synergism of at least about 25.

9. A composition according to claim 1, wherein the composition has a DPPH degree of synergism of at least about 51.

10. A composition according to claim 1, further comprising:
one or more citrus bioflavonoids; and
one or more fatty acids.

11. A composition according to claim 1, wherein said Indian gooseberry extract includes one or more low molecular weight hydrolysable tannins.

12. A composition according to claim 1, wherein the target component includes a target component selected from the group consisting of a superfruit; a fruit; a beverage; a nutritional product, a dietary supplement, a food product, a confectionary, an oil, a meal replacement, a cereal, a baked good, a candy, a gum, a lozenge, and any combinations thereof.

13. A composition according to claim 1, wherein the target component includes a superfruit and/or a fruit.

14. A composition according to claim 13, wherein said superfruit and/or fruit includes a superfruit and/or fruit selected from the group consisting of pomegranate, blueberry, cranberry, goji, acai, mangosteen, gac, noni, guarana, seabuckthorn, apple, orange, tomato, cranberry, concord grape, white grape, muscadine grape, grapefruit, strawberry, raspberry, blackberry, lemon, lime, key lime, kiwi, tangerine, banana, sweet cherry, black cherry, tart cherry, peach, plum, nectarine, pear, black currant, elderberry, apricot, mango, pineapple, passionfruit, yumberry, watermelon, cantaloupe, muskmelon, honeydew, starfruit, dragonfruit, damson plum, prune, raisin, lychee, schisandra, chokeberry, lingonberry, acerola, baobab, bilberry, camu camu, cupuacu, date, fig, durian, red guava, longan, papaya, Saskatoon berry, tamarind, yuzu, olive, and any combinations thereof.

15. A composition according to claim 1, wherein said target component includes a juice selected from the group consisting of filtered apple juice, unfiltered apple juice, cranberry juice, orange juice, tomato juice, pomegranate juice, blueberry juice, goji juice, acai juice, white grape, Concord grape, and any combinations thereof.

16. An antioxidant composition comprising:
about 19 wt. % to about 76 wt. % ascorbic acid;
about 0.3 wt. % to about 1.2 wt. % one or more citrus bioflavanoids;
about 0.1 wt. % to about 0.4 wt. % one or more fatty acids; and
about 12 wt. % to about 48 wt. % one or more low molecular weight hydrolysable tannins of an extract of Indian gooseberry.

17. A composition according to claim 16, further comprising:
about 2 wt. % to about 5 wt. % water; and
about 1.2 wt. % to about 4.8 wt. % sulfated ash.

18. A composition according to claim 16, comprising:
about 59 wt. % ascorbic acid;
about 0.9 wt. % one or more citrus bioflavanoids;
about 0.3 wt. % one or more fatty acids; and
about 22.5 wt. % one or more low molecular weight hydrolysable tannins of an extract of Indian gooseberry.

19. A composition according to claim 18, further comprising:
about 2.9 wt. % water; and
about 2.3 wt. % sulfated ash.

20. A method of synergistically increasing the antioxidant capability of a target component, the method comprising:
providing a target component;
adding a synergistically effective amount of an extract of Indian gooseberry, ascorbic acid, and one or more additional components to the target component such that the antioxidant capability of the target component is synergistically increased.
